(19) United States
(12) Patent Application Publication
(10) Pub. No.: US 2015/0217015 A1
(43) Pub. Date: Aug. 6, 2015

(54) LONG LASTING FRESHENING COMPOSITIONS

(71) Applicant: The Procter & Gamble Company, Cincinnati, OH (US)

(72) Inventors: Kristin Rhedrick WILLIAMS, West Chester, OH (US); Carla Jean COLINA, Cincinnati, OH (US); Michael David O'Young MUI, Cincinnati, OH (US); George Kavin MORGAN, III, Hamilton, OH (US); Steven Louis DIERSING, Cincinnati, OH (US); Rafael TRUJILLO, Mason, OH (US)

(73) Assignee: The Procter & Gamble Company, Cincinnati, OH (US)

(21) Appl. No.: 14/172,106

(22) Filed: Feb. 4, 2014

(51) Int. Cl. A61L 9/01

(52) U.S. Cl. CPC A61L 9/01

(57) ABSTRACT

Freshening compositions comprising a perfume material and a sulfur-containing pro-perfume; and methods thereof are provided. In some embodiments, the composition comprises dodecyl thio-damascone. Such freshening compositions may be used to prolong perfume release, while reducing malodors on inanimate surfaces or in the air.

### FIELD OF THE INVENTION

The present invention relates to long lasting freshening compositions comprising sulfur containing pro-perfumes such as thio-ketone and thio-damascone; and methods thereof.

### BACKGROUND OF THE INVENTION

Freshening products for reducing or masking malodors with scent on fabrics and in air are currently available and are described in the patent literature. Products having malodor reducing actives are also described in the patent literature. Research for new compositions that prolong freshness (e.g. scent) and malodor efficacy is ongoing.

There remains a need for compositions having long lasting freshening benefits, while reducing a broad range of malodors, including malodors caused by microbes, and while avoiding soiling and staining of fabrics that may be contacted by the freshening composition.

### SUMMARY OF THE INVENTION

In one embodiment, the freshening composition for reducing malodor comprises: a sulfur-containing pro-perfume; a perfume material; an effective amount of a malodor binding polymer; an aqueous carrier; wherein the ratio of said perfume materials to said sulfur-containing pro-perfume is about 2:1 to about 1:1.

In another embodiment, the freshening composition comprises: about 0.02% to about 0.1%, by weight of said composition, of a sulfur-containing pro-perfume; a perfume material; at least about 90%, by weight of said composition, of an aqueous carrier; wherein said composition is essentially free of any material that would soil or stain fabric.

In yet another embodiment, there is provided a freshening composition comprising: a sulfur-containing pro-perfume; a surfactant; an effective amount of a malodor binding polymer; about 90% or greater of aqueous carrier; wherein the ratio of said sulfur-containing pro-perfume to said surfactant is about 1:1 to about 1:10.

In yet another embodiment, there is provided a freshening composition comprising: a homopolymeric polyethyleneimine having a molecular weight of 1,000 to 2,000,000; a perfume material; about 0.02% to about 0.08%, by weight of said composition, of a dodecyl thio-damascone; about 90% to about 99.5%, by weight of said composition, of an aqueous carrier; wherein said composition is essentially free of any material that would soil or stain fabric.

The present invention also provides methods of reducing malodor comprising the steps of providing the composition disclosed herein and dispersing an effective amount of said composition onto an inanimate surface or into the air.

### BRIEF DESCRIPTION OF THE DRAWINGS

FIG. 1 is a graph showing the sensorial scent intensity of fabrics treated with freshening compositions according to the present invention in comparison to a freshening composition lacking a sulfur-containing pro-perfume.

### DETAILED DESCRIPTION OF THE INVENTION

The freshening composition of the present invention is designed to extend perfume release while reducing malodors. Such prolonged freshness and malodor reduction may last for at least about 24 hours, or at least about 48 hours after treating a space or surface with the freshening composition of the present invention.

In some embodiments, the freshening composition comprises a malodor binding polymer or counteractant that delivers a genuine malodor reduction. A genuine malodor reduction provides a sensory and analytically measurable (e.g. gas chromatograph) malodor reduction. Thus, if the freshening composition delivers a genuine malodor reduction, the freshening composition will neutralize malodors in the air and/or on fabrics. “Neutralize” or “neutralization” as used herein means chemically reacting with malodor components (e.g. the reaction of primary amines with aldehydes to form imines, reductive alkylation of amines, protonation and deprotonation of amines, polymerization or de-polymerization); or suppressing the volatility of malodorous components such that other parts of the composition may react (e.g. acid-base neutralization); or physically entrapping odorous molecules such that they are not re-released into the air (e.g. cyclodextrin inclusion complexes as described herein).

### I. Freshening Composition

The freshening composition of the present invention comprises a sulfur-containing pro-perfume, a perfume material, and an aqueous carrier.

A. Sulfur-Containing Pro-Perfume

The term “sulfur-containing pro-perfume” herein refers to a type of pro-perfume compound that contains sulfur. The term “pro-perfume” herein refers to compounds resulting from the reaction of perfume raw materials (“PRMs” or, singularly, “PRM”) with other chemicals, which have a covalent bond between one or more PRMs and these other chemicals.

The PRM is converted into a new material called a pro-perfume compound, which then may release the original PRM (i.e., pre-converted) upon exposure to a trigger such as water or light or atmospheric oxygen. Suitable pro-perfume compounds and methods of making the same can be found in U.S. Pat. Nos. 7,018,978; 6,861,402; 6,544,945; 6,093,691; 6,165,953; and 6,096,918.

The sulfur-containing pro-perfume herein may comprise a compound of formula (I):

Y—S-G-Q (I)
wherein:
   (i) Y is a radical selected from the group consisting of (Y-1) to (Y-7) shown herein below, including isomeric forms:

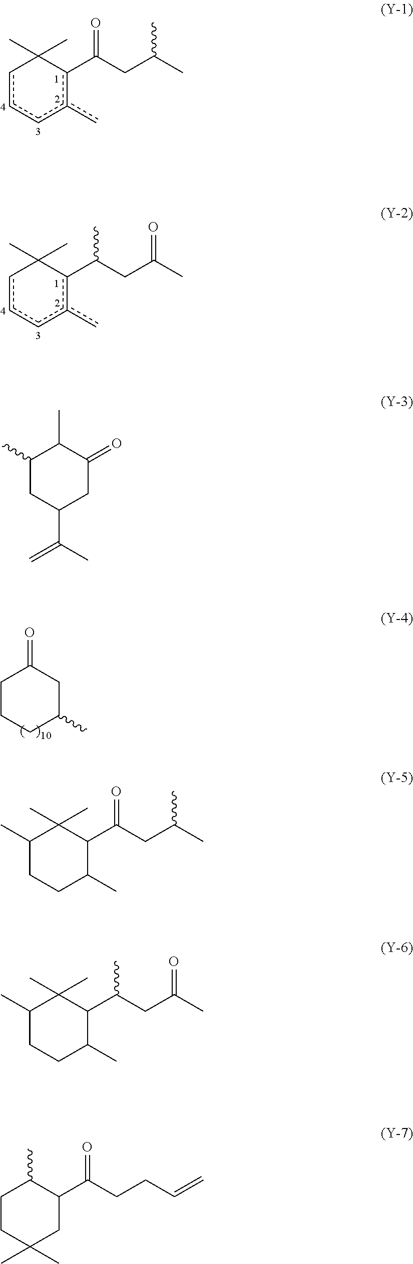

wherein the wavy lines represent the location of the sulfur bond, and the dotted lines represent a single or double bond;
(ii) G is selected from a divalent or trivalent radical derived from a linear or branched alkyl or alkenyl radical having from 2 to 15 carbon atoms; and
(iii) Q is selected from a hydrogen, a —S—Y group, or a —NR²—Y group, wherein Y is independently selected as defined above, and R² is selected from a hydrogen or a C₁-C₃ alkyl group.

In one embodiment, G is a divalent or trivalent radical, preferably a divalent radical derived from a linear or branched alkyl or alkenyl radical having from 2 to 15 carbon atoms, substituted with one or more groups selected from the group consisting of —OR¹, —NR¹₂, —COOR¹, R¹ groups, and a combination thereof, wherein R¹ is selected from a hydrogen or a C₁ to C₆ alkyl or alkenyl group. Preferably, G is a divalent radical derived from a linear or branched alkyl or alkenyl radical having from 2 to 15 carbon atoms, substituted with at least one —COOR¹ group, preferably substituted with a —COOR¹ group, wherein R¹ is selected from a hydrogen or a C₁ to C₆ alkyl or alkenyl group. Even more preferably, G is a divalent radical derived from a linear alkyl radical having a —CH₂CH(COOR¹) group, wherein R¹ is a hydrogen or a methyl or ethyl group. In an alternative embodiment, G is a divalent radical derived from a linear alkyl radical having from 8 to 15 carbon atoms which is either substituted or un-substituted.

In one embodiment, the sulfur-containing pro-perfume is a compound of formula (I) wherein Y is selected from Y-1, Y-2 or Y-3 groups as defined above, and G and Q are defined in any one of the above-described embodiments.

Preferably, the sulfur-containing pro-perfume is selected from the group consisting of methyl or ethyl 2-(4-oxo-4-(2,6,6-trimethylcyclohex-3-en-1-yl)butan-2-ylamino)-3-(4-oxo-4-(2,6,6-trimethylcyclohex-3-en-1-yl)butan-2-ylthio)propanate, methyl or ethyl 2-(4-oxo-4-(2,6,6-trimethylcyclohex-2-en-1-yl)butan-2-ylamino)-3-(4-oxo-4-(2,6,6-trimethylcyclohex-2-en-1-yl)butan-2-ylthio)propanate, methyl or ethyl 2-(2-oxo-4-(2,6,6-trimethylcyclohex-1-en-1-yl)butan-4-ylamino)-3-(2-oxo-4-(2,6,6-trimethylcyclohex-1-en-1-yl)butan-4-ylthio)propanate, methyl or ethyl 2-(2-oxo-4-(2,6,6-trimethylcyclohex-2-en-1-yl)butan-4-ylamino)-3-(2-oxo-4-(2,6,6-trimethylcyclohex-2-en-1-yl)butan-4-ylthio)propanate, 3-(dodecylthio)-1-(2,6,6-trimethylcyclohex-3-en-1-yl)-1-butanone, 3-(dodecylthio)-1-(2,6,6-trimethylcyclohex-2-en-1-yl)-1-butanone, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 2-dodecylsulfanyl-5-methyl-heptan-4-one, 2-cyclohexyl-1-dodecylsulfanyl-hept-6-en-3-one, 3-(dodecylthio)-5-isopropenyl-2-methylcyclohexanone, and a combination thereof.

More preferably, the sulfur-containing pro-perfume compound is selected from the group consisting of 3-(dodecylthio)-1-(2,6,6-trimethylcyclohex-3-en-1-yl)-1-butanone, 4-(dodecylthio)-4-(2,6,6-trimethylcyclohex-2-en1-yl)-2-butanone, 4-(dodecylthio)-4-(2,6,6-trimethylcyclohex-1-en-1-yl)-2-butanone and 3-(dodecylthio)-5-isopropenyl-2-methylcyclohexanone, and a combination thereof. 3-(dodecylthio)-1-(2,6,6-trimethylcyclohex-3-en-1-yl)-1-butanone is the most preferred sulfur-containing pro-perfume compound, such as Haloscent® D available from Firmenich located in Geneva, Switzerland.

The sulfur-containing pro-perfume compound can be present at any suitable level in the composition. Preferably, the sulfur-containing pro-perfume compound is present at least about 0.0001%, alternatively from about 0.0001% to about 3%, alternatively from about 0.0001% to about 1%, alternatively from about 0.01% to about 1%, alternatively from about 0.01% to about 0.1%, alternatively from about 0.02% to about 0.1%, alternatively from about 0.02% to about 0.08%, by weight of the composition.

In one embodiment, the freshening composition comprises dodecyl thio-damascone having the general structure shown below.

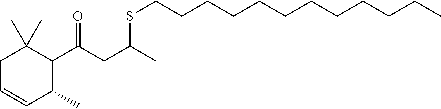 [SMILES: CCCCCCCCCCCCSC(C)CC(=O)C1[C@H](C)C=CCC1(C)C]

Thio-damascone may be present in an amount of about from about 0.0001% to about 1%, alternatively from about 0.01% to about 1%, alternatively from about 0.01% to about 0.1%, alternatively from about 0.02% to about 0.1%, alternatively from about 0.02% to about 0.08%, by weight of the composition. The ratio of perfume to thio-damascone may be about 2:1 to about 1:1, or about 1.5:1 to about 1:1, or about 1:1 to about 1:2, or about 1:1 to about 1:1.5 of the total perfume mixture.

B. PRMs

The composition of the present invention also includes PRMs. Any known PRM may be used.

PRMs Comprising Aldehyde or Ketone

A PRM comprising aldehyde or ketone may be incorporated in the composition herein to provide an immediate scent to users, without requiring any triggers to release the perfume. For example, each time when a user opens the container that contains a composition comprising such a PRM or applies the composition to a situs, the user would be able to smell a scent from the PRM. A wide variety of PRMs comprising aldehyde or ketone are suitable for use herein. The PRM comprising ketone is more preferred.

The PRM comprising ketone can comprise any PRMs which contain one or more ketone moieties and which can impart a desirable scent. The PRM may comprise ketone comprising a PRM selected from the group consisting of buccoxime; iso jasmone; methyl beta naphthyl ketone; musk indanone; tonalid/musk plus; alpha-damascone, beta-damascone, delta-damascone, iso-damascone, damascenone, damarose, methyl-dihydrojasmonate, menthone, carvone, camphor, fenchone, alpha-ionone, beta-ionone, dihydro-beta-ionone, gamma-methyl so-called ionone, fleuramone, dihydrojasmone, cis-jasmone, iso-e-super, methyl-cedrenyl-ketone or methyl-cedrylone, acetophenone, methyl-acetophenone, para-methoxy-acetophenone, methyl-beta-naphtyl-ketone, benzyl-acetone, benzophenone, para-hydroxy-phenyl-butanone, celery ketone or livescone, 6-isopropyldecahydro-2-naphtone, dimethyl-octenone, freskomenthe, 4-(1-ethoxyvinyl)-3,3,5,5,-tetramethyl-cyclohexanone, methyl-heptenone, 2-(2-(4-methyl-3-cyclohexen-1-yl)propyl)-cyclopentanone, 1-(p-menthen-6(2)-yl)-1-propanone, 4-(4-hydroxy-3-methoxyphenyl)-2-butanone, 2-acetyl-3,3-dimethyl-norbornane, 6,7-dihydro-1,1,2,3,3-pentamethyl-4(5h)-indanone, 4-damascol, dulcinyl or cassione, gelsone, hexalon, isocyclemone e, methyl cyclocitrone, methyl-lavender-ketone, orivon, para-tertiary-butyl-cyclohexanone, verdone, delphone, muscone, neobutenone, plicatone, veloutone, 2,4,4,7-tetramethyl-oct-6-en-3-one, tetrameran, hedione, floralozone, gamma undecalactone, ethylene brassylate, pentadecanolide, methyl nonyl ketone, cyclopentadecanone, cyclic ethylene dodecanedioate, 3,4,5,6-tetrahydropseudoionone, 8-hexadecenolide, dihydrojasmone, 5-cyclohexadecenone, and a combination thereof.

In one embodiment, the PRM comprising ketone comprises a PRM selected from the group consisting of alpha-damascone, delta-damascone, iso-damascone, carvone, gamma-methyl-ionone, beta-ionone, iso-e-super, 2,4,4,7-tetramethyl-oct-6-en-3-one, benzyl acetone, beta-damascone, damascenone, methyl dihydrojasmonate, methyl cedrylone, hedione, floralozone, and a combination thereof. Preferably, the PRM comprising ketone comprises delta-damascone.

The PRM comprising aldehyde can comprise any PRM which contains one or more aldehyde moieties and which can, like the PRM comprising ketone, also impart a desirable scent. Preferably, the PRM comprising aldehyde comprises a PRM selected from the group consisting of adoxal; anisic aldehyde; cymal; ethyl vanillin; florhydral; helional; heliotropin; hydroxycitronellal; koavone; lauric aldehyde; lyral; triplal, melonal, methyl nonyl acetaldehyde; p. t. bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-(para-methoxyphenyl propanal, 2-methyl-4-(2,6,6-trimethyl-2(1)-cyclohexen-1-yl) butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1h-indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carboxaldehyde, 5 or 6 methoxy0hexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, para-tolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl)benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, hexahydro-8,8-dimethyl-2-naphthaldehyde, 3-propyl-bicyclo[2.2.1]-hept-5-ene-2-carbaldehyde, 9-decenal, 2-decenal, 3-methyl-5-phenyl-1-pentanal, methylnonyl acetaldehyde, 1-p-menthene-q-carboxaldehyde, citral, lilial, cumin aldehyde, mandarin aldehyde, datilat, geranial, cyclamen aldehyde, capraldehyde, undecanal, lauraldehyde, nonaldehyde, 1,2-dodecenal, cis-8-undecen-1-al, tetrahydrogeranial, and a combination thereof.

In one embodiment, the PRM comprising aldehyde comprises a PRM selected from the group consisting of citral, 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, p-t-bucinal, lyral, cymal, methyl nonyl acetaldehyde, trans-2-nonenal, lilial, trans-2-nonenal, datilat, anisic aldehyde, geranial, i-octanal, helional, cuminaldehyde, triplal, melonal, and a combination thereof.

The PRM comprising aldehyde or ketone could be present at any suitable level in the composition. The PRM comprising aldehyde or ketone may be present from about 0.0001% to about 2%, alternatively from about 0.01% to about 4%, alternatively from about 0.1% to 1%, alternatively from about 0.5% to about 0.2%, by weight of the composition.

C. Malodor Binding Polymer The freshening composition of the present invention may include a malodor binding polymer. A malodor binding polymer is polymer having an available functional group (e.g. amine) that has the affinity to neutralize malodor components. Monomers having an available function group with an affinity to neutralize malodor components are also contemplated. In the case of amine based compounds, the amine will have an affinity for aldehyde malodors. The amine may react with aldehyde malodors and form a new compound, such as an aminol, imine, or enamine which is not odorous.

A malodor binding polymer may include amine based compounds, such as monoamines, amino acids, polyethyleneimine polymers (“PEIs”), modified PEIs, substituted PEIs; acrylic acid polymers, such as polyacrylate co-polymer (e.g. Acumer™ 9000 from Rohm & Haas), polyacrylic acid polymers (e.g. Acusol™ from Rohm & Haas), and modified acrylate copolymers (e.g. Aculyn™ from Rohm & Haas); and modified methacrylate copolymers (e.g. HydroSal™ from Salvona Technologies); or mixtures thereof.

Examples of suitable amino functional polymers containing at least one primary amine group for the purposes of the present invention are:

Polyvinylamine with a molecular weight (“MW”) of 300-2.10E6 Daltons (e.g Lupamine series 1500, 4500, 5000, 9000 available from BASF);

Polyvinylamine alkoxylated with a MW of ≧600 Daltons and a degree of ethoxylation of at least 0.5;

Polyvinylamine vinylalcohol—molar ratio 2:1, polyvinylaminevinylformamide—molar ratio 1:2 and polyvinylamine vinylformamide—molar ratio 2:1;

Triethylenetetramine, diethylenetriamine, tetraethylenepentamine;

Bis-aminopropylpiperazine;

amino substituted polyvinylalcohol with a MW ranging from 400-300,000 Daltons;

polyoxyethylene bis[amine] available from e.g. Sigma;

polyoxyethylene bis[6-aminohexyl] available from e.g. Sigma;

N,N′-bis-(3-aminopropyl)-1,3-propanediamine linear or branched (TPTA);

N,N′-bis-(3-aminopropyl)ethylenediamine;

bis(amino alkyl)alkyl diamine, linear or branched; and

1,4-bis-(3-aminopropyl)piperazine (BNPP).

Suitable amine based compounds include polyamino acids. Polyamino acids are made up of amino acids or chemically modified amino acids. The amino acids may be selected from cysteine, histidine, isoleucine, tyrosine, tryptophane, leucine, lysine, glutamic acid, glutamine, glycine, alanine, aspartic acid, arginine, asparagine, phenylalanine, proline, serine, histidine, threonine, methionine, valine, and mixtures thereof. Amino acid derivatives may be tyrosine ethylate, glycine methylate, tryptophane ethylate, or mixtures thereof; homopolymers of amino acids; hydroxyamines; polyamino acids; or mixtures thereof.

In chemically modified amino acids, the amine or acidic function of the amino acid has reacted with a chemical reagent. This is often done to protect these chemical amine and acid functions of the amino acid in a subsequent reaction or to give special properties to the amino acids, like improved solubility. Examples of such chemical modifications are benzyloxycarbonyl, aminobutyric acid, butyl ester, and pyroglutamic acid. More examples of common modifications of amino acids and small amino acid fragments can be found in the Bachem, 1996, Peptides and Biochemicals Catalog.

One polyamino acid is polylysine, alternatively polylysines or polyamino acids where more than 50% of the amino acids are lysine, since the primary amine function in the side chain of the lysine is the most reactive amine of all amino acids. One polyamino acid has a MW of 500 to 10,000,000, alternatively between 2000 and 25,000.

The polyamino acid can be cross linked. Cross linking can be obtained for example by condensation of the amine group in the side chain of the amino acid like lysine with the carboxyl function on the amino acid or with protein cross linkers like PEG derivatives. The cross linked polyamino acids still need to have free primary and/or secondary amino groups left for neutralization. Cross linked polyamino acid has a MW of 20,000 to 10,000,000; alternatively between 200,000 and 2,000,000.

The polyamino acid or the amino acid can be co-polymerized with other reagents like for instance with acids, amides, acyl chlorides, aminocaproic acid, adipic acid, ethylhexanoic acid, caprolactam, or mixtures thereof. The molar ratio used in these copolymers ranges from 1:1 (reagent/amino acid (lysine)) to 1:20, alternatively from 1:1 to 1:10. The polyamino acid like polylysine can be unethoxylated or partially ethoxylated so long as the requisite amount of primary amine remains in the polymer.

In one embodiment, the malodor binding polymer is a PEI. It has been surprisingly discovered that amine based polymers at a pH of about 4 to about 8, alternatively above 5 to about 8, alternatively 7 can neutralize amine based odors. PEIs have the following general formula:

—(CH2-CH2-NH)n-; n=10-105

Homopolymeric PEIs are branched, spherical polyamines with a well defined ratio of primary, secondary and tertiary amine functions. They are best described in the following partial structural formula:

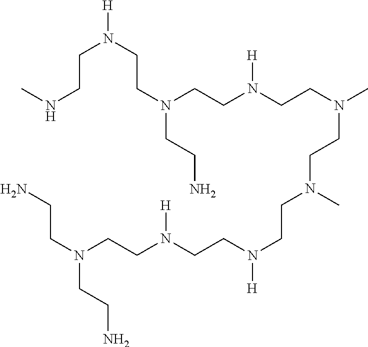

The chemical structure of homopolymeric PEIs follows a simple principle: one amine function—two carbons.

The freshening composition may comprise a homopolymeric polyethylenimine having a MW of about 800 to about 2,000,000, alternatively about 1,000 to about 2,000,000, alternatively about 1,200 to about 25,000, alternatively about 1,300 to about 25,000, alternatively about 2,000 to about 25,000, alternatively about 10,000 to about 2,000,000, alternatively about 25,000 to about 2,000,000, alternatively about 25,000. Exemplary homopolymeric PEIs include those that are commercially available under the tradename Lupasol® from BASF. Lupasol products are usually obtained through polymerization of the ethylenimine monomer. The ethylenimine monomer has totally reacted in the polymer matrix. Suitable Lupasol products include Lupasol FG (MW 800), G20wfv (MW 1300), PR8515 (MW 2000), WF (MW 25,000), FC (MW 800), G20 (MW 1300), G35 (MW 1200), G100 (MW 2000), HF (MW 25,000), P (MW 750,000), PS (MW 750,000), SK (MW 2,000,000), SNA (MW 1,000,000).

In some embodiments, the freshening composition comprises Lupasol HF or WF (MW 25,000), P (MW 750,000), PS (MW 750,000), SK (MW 2,000,000), 620wfv (MW 1300) or PR 1815 (MW 2000), or Epomin SP-103, Epomin SP-110, Epomin SP-003, Epomin SP-006, Epomin SP-012, Epomin SP-018, Epomin SP-200, or partially alkoxylated polyethyleneimine, like polyethyleneimine 80% ethoxylated from Aldrich. In one embodiment, the freshening composition contains Lupasol WF (MW 25,000).

Also suitable amine based compounds for use in the freshening composition are modified PEIs, partially alkylated polyethylene polymers, PEIs with hydroxyl groups, 1,5-pentanediamine, 1,6-hexanediamine, 1,3 pentanediamine, 3-dimethylpropanediamine, 1,2-cyclohexanediamine, 1,3-bis(aminomethyl)cyclohexane, tripropylenetetraamine, bis(3-aminopropyl)piperazine, dipropylenetriamine, tris(2-aminoethylamine), tetraethylenepentamine, bishexamethylenetriamine, bis(3-aminopropyl) 1,6-hexamethylenediamine, 3,3′-diamino-N-methyldipropylamine, 2-methyl-1,5-pentanediamine, N,N,N′,N′-tetra(2-aminoethyl)ethylenediamine, N,N,N′,N′-tetra(3-aminopropyl)-1,4-butanediamine, pentaethylhexamine, 1,3-diamino-2-propyl-tert-butylether, isophorondiamine, 4,4′,-diaminodicyclohylmethane, N-methyl-N-(3-aminopropyl)ethanolamine, spermine, spermidine, 1-piperazineethaneamine, 2-(bis(2-aminoethyl)amino)ethanol, ethoxylated N-(tallowalkyl)trimethylene diamines, poly[oxy(methyl-1,2-ethanediyl)], α-(2-aminomethyl-ethoxy)- (=C.A.S No. 9046-10-0); poly[oxy(methyl-1,2-ethanediyl)], α-hydro-)-ω-(2-aminomethylethoxy)-, ether with 2-ethyl-2-(hydroxymethyl)-1,3-propanediol (=C.A.S. No. 39423-51-3); commercially available under the tradename Jeffamines T-403, D-230, D-400, D-2000; 2,2′,2″-triaminotriethylamine; 2,2′-diamino-diethylamine; 3,3′-diamino-dipropylamine, 1,3 bis aminoethyl-cyclohexane commercially available from Mitsubishi, and the C12 Sternamines commercially available from Clariant like the C12 Sternamin(propylenamine)ₙ with n=3/4.

In one embodiment, the malodor binding polymer may be used in an effective amount to provide a reduction of microbes on fabric and/or in the air. When using a malodor binding polymer, an effective amount reduces microbes by at least 1 log difference as compared to a composition lacking the malodor binding polymer. This difference is then attributed to the use of the malodor binding polymer and not the inherent variability in the microbial species.

Suitable levels of malodor binding polymer are from about 0.01% to about 2%, alternatively from about 0.01% to about 1%, alternatively about 0.01% to about 0.8%, alternatively about 0.01% to about 0.6%, alternatively about 0.01% to about 0.1%, alternatively about 0.01% to about 0.07%, alternatively about 0.07%, by weight of the freshening composition. Compositions with higher amount of malodor binding polymer may make fabrics susceptible to soiling and/or leave unacceptable visible stains on fabrics as the solution evaporates off of the fabric.

D. Malodor Counteractants

The freshening composition may, optionally, utilize one or more malodor counteractants. Malodor counteractants may include components which lower the vapor pressure of odorous compounds, solubilize malodor compounds, physically entrap odors (e.g. flocculate or encapsulate), chemically react, physically bind odors, or physically repel odors from binding to inanimate surfaces.

1. Low Molecular Weight Polyols

Low molecular weight polyols with relatively high boiling points, as compared to water, such as ethylene glycol, diethylene glycol, triethylene glycol, propylene glycol, dipropylene glycol, and/or glycerine may be utilized as a malodor counteractant for improving odor neutralization of the freshening composition of the present invention. Some polyols, e.g., dipropylene glycol, are also useful to facilitate the solubilization of some perfume ingredients in the composition of the present invention.

The glycol used in the freshening composition of the present invention may be glycerine, ethylene glycol, propylene glycol, dipropylene glycol, polyethylene glycol, propylene glycol methyl ether, propylene glycol phenyl ether, propylene glycol methyl ether acetate, propylene glycol n-butyl ether, dipropylene glycol n-butyl ether, dipropylene glycol n-propyl ether, ethylene glycole phenyl ether, diethylene glycol n-butyl ether, dipropylene glycol n-butyl ether, diethylene glycol mono butyl ether, dipropylene glycol methyl ether, tripropylene glycol methyl ether, tripropylene glycol n-butyl ether, other glycol ethers, or mixtures thereof. In one embodiment, the glycol used is ethylene glycol, propylene glycol, or mixtures thereof. In another embodiment, the glycol used is diethylene glycol.

Typically, the low molecular weight polyol is added to the composition of the present invention at a level of from about 0.01% to about 5%, by weight of the composition, alternatively from about 0.05% to about 1%, alternatively from about 0.1% to about 0.5%, by weight of the composition. Compositions with higher concentrations may make fabrics susceptible to soiling and/or leave unacceptable visible stains on fabrics as the solution evaporates off of the fabric. The weight ratio of low molecular weight polyol to the malodor binding polymer is from about 500:1 to about 4:1, alternatively from about 1:100 to about 25:1, alternatively from about 1:50 to about 4:1, alternatively about 4:1.

2. Cyclodextrin

In some embodiments, the freshening composition may include solubilized, water-soluble, uncomplexed cyclodextrin. As used herein, the term “cyclodextrin” includes any of the known cyclodextrins such as unsubstituted cyclodextrins containing from six to twelve glucose units, especially alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms, therefore this surface is fairly hydrophobic. The unique shape and physical-chemical property of the cavity enable the cyclodextrin molecules to absorb (form inclusion complexes with) organic molecules or parts of organic molecules which can fit into the cavity. Many perfume molecules can fit into the cavity.

Cyclodextrin molecules are described in U.S. Pat. No. 5,714,137, and U.S. Pat. No. 5,942,217. Suitable levels of cyclodextrin are from about 0.1% to about 5%, alternatively from about 0.2% to about 4%, alternatively from about 0.3% to about 3%, alternatively from about 0.4% to about 2%, by weight of the freshening composition. Freshening compositions with higher concentrations can make fabrics susceptible to soiling and/or leave unacceptable visible stains on fabrics as the solution evaporates off of the fabric. The latter is especially a problem on thin, colored, synthetic fabrics. In order to avoid or minimize the occurrence of fabric staining, the fabric may be treated at a level of less than about 5 mg of cyclodextrin per mg of fabric, alternatively less than about 2 mg of cyclodextrin per mg of fabric.

E. Buffering Agent

The freshening composition of the present invention may, optionally, include a buffering agent which may be a dibasic acid, carboxylic acid, or a dicarboxylic acid like maleic acid. The acid may be sterically stable, and used in this composition solely for maintaining the desired pH. The freshening composition may have a pH from about 4 to about 8, alternatively from about 5 to about 8, alternatively from about 5 to about 7, alternatively about 7, alternatively about 6.6.

Carboxylic acids such as citric acid may act as metal ion chelants and can form metallic salts with low water solubility. As such, in some embodiments, the freshening composition is essentially free of citric acids. The buffer can be alkaline, acidic or neutral.

Other suitable buffering agents for freshening compositions of this invention include biological buffering agents. Some examples are nitrogen-containing materials, sulfonic acid buffers like 3-(N-morpholino)propanesulfonic acid (MOPS) or N-(2-Acetamido)-2-aminoethanesulfonic acid (ACES), which have a near neutral 6.2 to 7.5 pKa and provide adequate buffering capacity at a neutral pH. Other examples are amino acids such as lysine or lower alcohol amines like mono-, di-, and tri-ethanolamine. Other nitrogen-containing buffering agents are tri(hydroxymethyl)amino methane (HOCH2)3CNH3 (TRIS), 2-amino-2-ethyl-1,3-propanediol, 2-amino-2-methyl-propanol, 2-amino-2-methyl-1,3-propanol, disodium glutamate, N-methyl diethanolamide, 2-dimethylamino-2-methylpropanol (DMAMP), 1,3-bis(methylamine)-cyclohexane, 1,3-diamino-propanol N,N′-tetra-methyl-1,3-diamino-2-propanol, N,N-bis(2-hydroxyethyl)glycine (bicine) and N-tris(hydroxymethyl)methyl glycine (tricine). Mixtures of any of the above are also acceptable.

The freshening compositions may contain at least about 0%, alternatively at least about 0.001%, alternatively at least about 0.01%, by weight of the composition, of a buffering agent. The composition may also contain no more than about 1%, alternatively no more than about 0.75%, alternatively no more than about 0.5%, by weight of the composition, of a buffering agent.

F. Solubilizer

The freshening composition of the present invention may, optionally, contain a solubilizing aid to solubilize any excess hydrophobic organic materials, particularly any PRMs, and also optional ingredients (e.g., insect repelling agent, antioxidant, etc.) which can be added to the composition, that are not readily soluble in the composition, to form a clear solution. A suitable solubilizing aid is a surfactant, such as a no-foaming or low-foaming surfactant. Suitable surfactants are anionic surfactants, nonionic surfactants, cationic surfactants, amphoteric surfactants, zwitterionic surfactants, and mixtures thereof.

In some embodiments, the freshening composition contains nonionic surfactants, cationic surfactants, and mixtures thereof. In one embodiment, the freshening composition contains hydrogenated castor oil. One suitable hydrogenated castor oil that may be used in the present composition is Basophor™, available from BASF.

Compositions containing anionic surfactants and/or detergent surfactants may make fabrics susceptible to soiling and/or leave unacceptable visible stains on fabrics as the solution evaporates off of the fabric. In some embodiments, the freshening composition is free of anionic surfactants and/or detergent surfactants.

When the solubilizing agent is present, it is typically present at a level of from about 0.01% to about 3%, alternatively from about 0.05% to about 1%, alternatively from about 0.01% to about 0.05%, by weight of the freshening composition. Freshening compositions with higher concentrations may make fabrics susceptible to soiling and/or leave unacceptable visible stains on fabrics as the solution evaporates off of the fabric.

G. Antimicrobial Compounds

The freshening composition of the present invention may, optionally, include an effective amount of a compound for reducing microbes in the air or on inanimate surfaces. Antimicrobial compounds are effective on gram negative and gram positive bacteria and fungi typically found on indoor surfaces that have contacted human skin or pets such as couches, pillows, pet bedding, and carpets. Such microbial species include Klebsiella pneumoniae, Staphylococcus aureus, Aspergillus niger, Klebsiella pneumoniae, Steptococcus pyogenes, Salmonella choleraesuis, Escherichia coli, Trichophyton mentagrophytes, and Pseudomonoas aeruginosa. In some embodiments, the antimicrobial compounds are also effective on viruses such H1-N1, Rhinovirus, Respiratory Syncytial, Poliovirus Type 1, Rotavirus, Influenza A, Herpes simplex types 1 & 2, Hepatitis A, and Human Coronavirus.

Antimicrobial compounds suitable in the freshening composition of the present invention can be any organic material which will not cause damage to fabric appearance (e.g., discoloration, coloration such as yellowing, bleaching). Water-soluble antimicrobial compounds include organic sulfur compounds, halogenated compounds, cyclic organic nitrogen compounds, low molecular weight aldehydes, quaternary compounds, dehydroacetic acid, phenyl and phenoxy compounds, or mixtures thereof.

In one embodiment, a quaternary compound is used. Examples of commercially available quaternary compounds suitable for use in the freshening composition is Barquat® available from Lonza Corporation; and didecyl dimethyl ammonium chloride quat under the trade name Bardac® 2250 from Lonza Corporation.

The antimicrobial compound may be present in an amount from about 500 ppm to about 7000 ppm, alternatively from about 1000 ppm to about 5000 ppm, alternatively from about 1000 ppm to about 3000 ppm, alternatively from about 1400 ppm to about 2500 ppm, by weight of the freshening composition.

H. Preservatives

The freshening composition of the present invention may, optionally, include a preservative. The preservative is included in the present invention in an amount sufficient to prevent spoilage or prevent growth of inadvertently added microorganisms for a specific period of time, but not sufficient enough to contribute to the odor neutralizing performance of the freshening composition. In other words, the preservative is not being used as the antimicrobial compound to kill microorganisms on the surface onto which the composition is deposited in order to eliminate odors produced by microorganisms. Instead, it is being used to prevent spoilage of the freshening composition in order to increase the shelf-life of the composition.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diaol materials, isothiazolinones, quaternary compounds, benzoates, low molecular weight alcohols, dehydroacetic acid, phenyl and phenoxy compounds, or mixtures thereof.

Non-limiting examples of commercially available water-soluble preservatives for use in the present invention include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl)2,5-dioxo-4-imidazolidinyl]-N,N′-bis(hydroxy-methyl)urea, commonly known as diazolidinyl urea, available under the trade name Germall® II from Sutton Laboratories, Inc.; N,N″-methylenebis {N′-[1-(hydroxymethyl)-2,5-dioxo-4-imidazolidinyl]urea}, commonly known as imidazolidinyl urea, available, e.g., under the trade name Abiol® from 3V-Sigma; Unicide U-13® from Induchem; Germall 115® from Sutton Laboratories, Inc.; polymethoxy bicyclic oxazolidine, available under the trade name Nuosept® C from Hills America; formaldehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation.

Suitable levels of preservative are from about 0.0001% to about 0.5%, alternatively from about 0.0002% to about 0.2%, alternatively from about 0.0003% to about 0.1%, by weight of the freshening composition.

I. Wetting Agent

The freshening composition may, optionally, include a wetting agent that provides a low surface tension that permits the composition to spread readily and more uniformly on hydrophobic surfaces like polyester and nylon. It has been found that the aqueous solution, without such a wetting agent will not spread satisfactorily. The spreading of the composition also allows it to dry faster, so that the treated material is ready to use sooner. Furthermore, a composition containing a wetting agent may penetrate hydrophobic, oily soil better for improved malodor neutralization. A composition containing a wetting agent may also provide improved “in-wear” electrostatic control. For concentrated compositions, the wetting agent facilitates the dispersion of many actives such as antimicrobial actives and perfumes in the concentrated aqueous compositions.

Non-limiting examples of wetting agents include block copolymers of ethylene oxide and propylene oxide. Suitable block polyoxyethylene-polyoxypropylene polymeric surfactants include those based on ethylene glycol, propylene glycol, glycerol, trimethylolpropane and ethylenediamine as the initial reactive hydrogen compound. Polymeric compounds made from a sequential ethoxylation and propoxylation of initial compounds with a single reactive hydrogen atom, such as C₁₂₋₁₈ aliphatic alcohols, are not generally compatible with the cyclodextrin. Certain of the block polymer surfactant compounds designated Pluronic® and Tetronic® by the BASF-Wyandotte Corp., Wyandotte, Mich., are readily available.

Non-limiting examples of cyclodextrin-compatible wetting agents of this type are described in U.S. Pat. No. 5,714,137 and include the Silwet® surfactants available from Momentive Performance Chemical, Albany, N.Y. Exemplary Silwet surfactants are as follows:

| Name | Average MW |
|---|---|
| L-7608 | 600 |
| L-7607 | 1,000 |
| L-77 | 600 |
| L-7605 | 6,000 |
| L-7604 | 4,000 |
| L-7600 | 4,000 |
| L-7657 | 5,000 |
| L-7602 | 3,000; |

and mixtures thereof.

The total amount of surfactants (e.g. solubilizer, wetting agent) in the freshening composition is from 0% to about 3% or no more than 3%, alternatively from 0% to about 1% or no more than 1%, alternatively from 0% to about 0.9% or no more than 0.9%, alternatively from 0% to about 0.7 or no more than 0.7%, alternatively from 0% to about 0.5% or no more than 0.5%, alternatively from 0% to 0.3% or no more than about 0.3%, by weight of the composition. Compositions with higher concentrations can make fabrics susceptible to soiling and/or leave unacceptable visible stains on fabrics as the solution evaporates.

In some embodiments, the ratio of sulfur-containing pro-perfume to total surfactant is from about 1:1 to about 1:10, or from about 1:1 to about 1:6, or from about 1:1 to about 1:4; or from about 1:4, or about 1:1.

J. Aqueous Carrier

The freshening composition of the present invention includes an aqueous carrier. The aqueous carrier which is used may be distilled, deionized, or tap water. Water may be present in any amount for the composition to be an aqueous solution. In some embodiments, water may be present in an amount from about 85% to 99.5%, alternatively from about 90% to about 99.5%, alternatively from about 92% to about 99.5%, alternatively from about 95%, by weight of said freshening composition. Water containing a small amount of low molecular weight monohydric alcohols (e.g., ethanol, methanol, and isopropanol, or polyols, such as ethylene glycol and propylene glycol) can also be useful. However, the volatile low molecular weight monohydric alcohols such as ethanol and/or isopropanol should be limited since these volatile organic compounds will contribute both to flammability problems and environmental pollution problems. If small amounts of low molecular weight monohydric alcohols are present in the composition of the present invention due to the addition of these alcohols to such things as perfumes and as stabilizers for some preservatives, the level of monohydric alcohol may be less than about 6%, alternatively less than about 3%, alternatively less than about 1%, by weight of the freshening composition.

K. Other Optional Ingredients

Adjuvants can be optionally added to the freshening composition herein for their known purposes. Such adjuvants include, but are not limited to, water soluble metallic salts, antistatic agents, insect and moth repelling agents, colorants, antioxidants, and mixtures thereof.

### II. Method of Making

The freshening composition can be made in any suitable manner known in the art. All of the ingredients can simply be mixed together. In certain embodiments, it may be desirable to make a concentrated mixture of ingredients and dilute by adding the same to an aqueous carrier before dispersing the composition into the air or on an inanimate surface. In another embodiment, the malodor binding polymer may be dispersed in one vessel containing deionized water and ethanol, and low molecular polyols. To this vessel, then, the buffer is added until fully dispersed and visually dissolved. In a separate vessel, the solubilizer and perfume are mixed until homogenous. The solution of solubilizer and perfume are then added to the first mixing vessel, and mixed until homogenous.

### III. Methods of Use

The freshening composition of the present invention can be used by dispersing, e.g., by placing the aqueous solution into a dispensing means, such as a spray dispenser and spraying an effective amount into the air or onto the desired surface or article. “Effective amount”, when used in connection with the amount of the freshening composition, means an amount sufficient to provide at least about 24 hours of freshness or scent to the treated air, surface, or article, yet not so much as to saturate or create a pool of liquid on an article or surface and so that, when dry, there is no visual deposit readily discernible. Where malodor reducing ingredients are included, “effective amount”, when used in connection with the amount of the freshening composition, means an amount that provides the foregoing and also provides neutralization of a malodor to the point that it is not discernible by the human sense of smell, yet not so much as to saturate or create a pool of liquid on an article or surface and so that, when dry, there is no visual deposit readily discernible. Dispersing can be achieved by using a spray device, a roller, a pad, etc.

A variety of surfaces and articles can be treated with the freshening composition of the present invention. Suitable surfaces and articles include household surfaces selected from the group consisting of countertops, cabinets, walls, floors, bathroom surfaces, and kitchen surfaces; fabric and/or fabric articles including clothes, curtains, drapes, upholstered furniture, carpeting, bed linens, bath linens, tablecloths, sleeping bags, tents, car interior, car carpet, fabric car seats; shoes; shower curtains; garbage cans and/or recycling bins; major household appliances including refrigerators, freezers, washing machines, automatic dryers, ovens, microwave ovens, dishwashers, etc.; cat litter, pet bedding and pet houses.

### Examples

### Effect of Thio-damascone on Perfume Release

### Formulations:

TABLE 1

| Ingredient | DOX1 | DOX2 | DOX3 | DOX4 | DOX5 |
|---|---|---|---|---|---|
| DI Water | 94.992 | 94.972 | 94.967 | 94.982 | 95.047 |
| Ethanol | 3 | 3 | 3 | 3 | 3 |
| Lupasol ® WF | 0.065 | 0.065 | 0.065 | 0.065 | 0 |
| (CAS 9002-98-6) | | | | | |
| Diethylene Glycol | 0.175 | 0.175 | 0.175 | 0.175 | 0.175 |
| Silwet L-7600 | 0.1 | 0.1 | 0.1 | 0.1 | 0.1 |
| Uniquat | 0.1 | 0.1 | 0.1 | 0.1 | 0.1 |
| Maleic Acid | 0.05 | 0.05 | 0.05 | 0.05 | 0.05 |
| Citric Acid | 0.015 | 0.015 | 0.015 | 0.015 | 0.015 |
| Perfume mixture 1 | 0.143 | 0.143 | 0.143 | 0.143 | 0.143 |
| Haloscent-D ™ | | 0.02 | | | |
| thio-damasone | | | | | |
| Perfume pre-mix with | | | 0.025 | | |
| Delta Damascone at | | | | | |
| 40% (by wt of mixture 2) | | | | | |
| Delta Damascone | | | | 0.01 | 0.01 |
| Aquasolved ™ (from | 0.6 | 0.6 | 0.6 | 0.6 | 0.6 |
| Firmenich)/Solvent | | | | | |
| Basophor ELH60 | 0.125 | 0.125 | 0.125 | 0.125 | 0.125 |
| Hydroxypropyl Beta CD | 0.63 | 0.63 | 0.63 | 0.63 | 0.63 |
| Sodium Hydroxide | 0.005 | 0.005 | 0.005 | 0.005 | 0.005 |

### GC/MS Procedure:

Formulations according to Table 1 are prepared. Each formulation is placed in a pre-compression trigger bottle. The spray for each fabric swatch consists of 3 full strokes (unless noted differently) of the bottle. The bottle is held 6 inches away from the fabric swatch and the spray is centered on the fabric swatch. After the time period specified, each fabric swatch is cut in half, rolled, and placed into a 125 ml headspace vial. The vials are sealed. The vials are allowed to equilibrate for at least 2 hours at 100° C. and then analyzed by sampling each vial using a PDMS SPME fiber and analyzed by GC/MS. Perfume components previously identified, are then tracked through all the samples. Data is compiled of total area count of the cumulative area counts of the individual peaks (unless noted differently).

### GC/MS Data:

Table 2 shows that Dox 2, containing a thio-damascone, performs better than Dox 1, 3, 4, and 5 formulations.

TABLE 2

| | Days | | | |
|---|---|---|---|---|
| | 0 | 1 | 3 | 7 |
| Dox 1 | 792199.8 | 7450.75 | 4998.5 | 3309.25 |
| Dox 2 | 1715780 | 1676194 | 716662 | 48505.75 |
| Dox 3 | 10611728 | 156580.3 | 66790.33 | 21687.5 |
| Dox 4 | 4799732 | 112571.5 | 63832.75 | 17330.25 |
| Dox 5 | 7637442 | 118412 | 39187.25 | 17974.5 |

All values in Table 2 are FID (Flame Ionization Detector) area count for D-Damascone.

### Intensity and Longevity Sensorial Evaluation

### Formulations:

TABLE 3

| Ingredient | Composition 1 | Composition 2 |
|---|---|---|
| Lupasol ® WF | 0.065 | 0.065 |
| (CAS 9002-98-6) | | |
| Diethylene Glycol | 0.175 | 0.175 |
| dodecyl thio-damascone | 0.1 | 0 |
| Perfume mixture | 0.14 | 0.14 |
| Hydroxypropyl Beta CD | 0.63 | 0.63 |
| Uniquat 2250 | 0.06 | 0.06 |
| Silwet L-7600 | 0.10 | 0.10 |
| Citric Acid | 0.015 | 0.015 |
| Maleic Acid | 0.03 | 0.03 |
| (CAS 110-16-7) | | |
| Sodium Hydroxide | As needed to | As needed to |
| | adjust pH | adjust pH |
| Koralone B-119 | 0.015 | 0.015 |
| Ethanol | 3.0 | 3.0 |
| Deionized Water | To 100 | To 100 |
| Total | 100 | 100 |

### General Test Design:

Panelists: All evaluations discussed herein are assessments by expert panelist who are screened for their olfactive acuity. Expert panelists are calibrated quarterly and meet the ASTM no. E-544 standards for sensitivity and reproducibility. The ASTM is the premier scientific body within the US for establishing standards for the testing of materials.
Data: All data generated demonstrates statistically significant differences at a minimum of 95% confidence level.

### Preparation of Fabric Swatches:

Fabric swatches are prepared under a fume hood. Clean swatches are used, in this case clean stripped 100% cotton terry towels.
The fabric swatches are removed from a mylar bag and each swatch is labeled with an assigned three digit blinding code or one letter designation, and are placed back in specifically labeled mylar bags until testing begins.

### Panelist Procedure:

Panelists evaluate swatches in groups of 2-5 depending on the total number of panelists available. There are always 4 groups per test.
Each panelist receives a ballot with the specified test codes and evaluation order.

### Treatment Procedure:

A total of 8 fabric swatches are sprayed, 4 with each one of Composition 1 or 2 (see Table 3). The swatches are sprayed from a distance of 6 inches. One swatch at a time is sprayed.
After treatment, each fabric swatch is placed on a garment rack.

### Evaluations:

Panelists in assigned groups evaluate swatches for designated attribute e.g. perfume intensity for the initial time point.
Fabrics are presented in a blinded randomized order
Panelists mark their ballots by evaluating the attribute on a 0-100 scale.

| | |
|---|---|
| 0 | No perfume present. |
| 10 | I think there is perfume present (unsure). |
| 20 | I detect something, but can I recognize it? |
| 25 | Slight perfume present. |
| 50 | Moderate perfume present. |
| 75 | Strong perfume present. |

### Results:

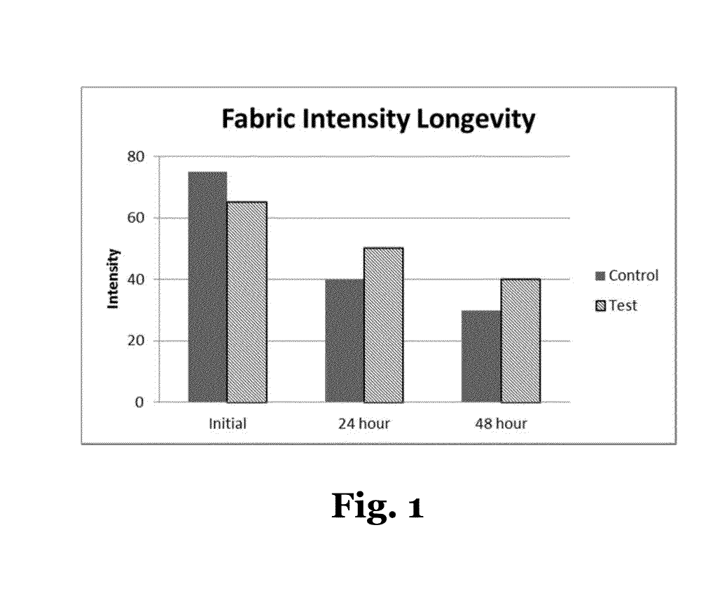

FIG. 1 shows a noticeable increase in fabric scent intensity at 24 and 48 hour increments.

### Stability Test

### Formulations:

TABLE 4

| | CONTROL | Test 1 | Test 2 | Test 3 |
|---|---|---|---|---|
| | Perfume | Perfume + | Perfume + | nil polymer + |
| Ingredient | only | Haloscent-D | Damascone | nil Damascone |
| DI Water | 95.915 | 95.815 | 95.865 | 95.895 |
| Ethanol | 3.000 | 3.000 | 3.000 | 3.000 |
| Lupasol HF | 0.0650 | 0.0650 | 0.0650 | 0.0000 |
| Diethylene Glycol | 0.175 | 0.175 | 0.175 | 0.175 |
| Silwet L-7600 | 0.100 | 0.100 | 0.100 | 0.100 |
| Uniquat 2250 | 0.060 | 0.060 | 0.060 | 0.060 |
| Maleic Acid | 0.050 | 0.050 | 0.050 | 0.050 |
| Citric Acid | 0.015 | 0.015 | 0.015 | 0.015 |
| Perfume Premix*: | | | | |
| Fragrance | 0.140 | 0.140 | 0.140 | 0.140 |
| Damascone | | 0.000 | 0.050 | 0.050 |
| Haloscent-D | | 0.100 | | |
| Solvent (low MW glycols) | 0.350 | 0.350 | 0.350 | 0.350 |
| Basophor ELH60 | 0.125 | 0.125 | 0.125 | 0.125 |
| Hydroxypropyl Beta CD | 0.000 | | 0.000 | 0.000 |
| Sodium Hydroxide | 0.005 | 0.005 | 0.005 | 0.040 |
| TOTAL | 100 | 100 | 100 | 100 |

### Protocol:

Test formulations were prepared according to Table 4. Samples of each test formulation are individually placed in 8 oz. glass jars and visually assessed for color. Samples prepared are initially clear and over time, if damascone is released and free in the formulation, an undesirable Schiff-base reaction occurs creating discoloration, turning the solution yellow.

To illustrate the differences in the samples prepared, Hunter L-a-b measurements were obtained via a HunterLab LabScan XE spectrophotometer to quantitatively describe the differences between samples. The Hunter L-a-b color space is organized in a cube form. The L-axis runs from top to bottom. The maximum value for L is 100 and the minimum value is zero, which would be black. The a and b axes have no specific numerical limits. Positive a is red, while negative a is green. Positive b is yellow, while negative b is blue. The data below shows the most notable shift from clear to yellow, which is noted as a positive b value in the Hunter L-a-b scale.

### Results:

TABLE 5

| | Week 1 | Week 12 | % Change |
|---|---|---|---|
| | L-a-b value | L-a-b value | Over Time |
| Samples at 25° C. | | | |
| Control | 1.29 | 1.43 | 10.6 |
| Test 1 | 0.89 | 1.07 | 20.0 |
| Test 2 | 0.97 | 1.14 | 17.7 |
| Test 3 | 1.03 | 1.13 | 9.7 |
| Samples at 40° C. | | | |
| Control | 0.68 | 0.41 | −39.6 |
| Test 1 | 0.75 | 0.69 | −8.3 |
| Test 2 | 0.76 | 0.38 | −50.4 |
| Test 3 | 0.77 | 0.31 | −59.8 |

Table 5 demonstrates, at 25° C. and 40° C., thio-damascone releases less damascone versus all other test legs.

All percentages stated herein are by weight unless otherwise specified. It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A freshening composition for reducing malodor comprising:
a sulfur-containing pro-perfume;
a perfume material;
an effective amount of a malodor binding polymer;
an aqueous carrier;
wherein the ratio of said perfume materials to said sulfur-containing pro-perfume is about 2:1 to about 1:1.

2. The composition of claim 1, wherein said sulfur-containing pro-perfume is present in an amount from about 0.01% to about 1.5%, by weight of said composition

3. The composition of claim 1, wherein said sulfur-containing pro-perfume is present in an amount from about 0.02% to about 0.1%, by weight of said composition

4. The composition of claim 1, wherein said ratio of perfume materials to sulfur-containing pro-perfume is about 1:1.

5. The composition of claim 1, wherein said sulfur-containing pro-perfume is a C4-C12 thio-damascone.

6. The composition of claim 1, wherein said sulfur-containing pro-perfume is dodecyl thio-damascone.

7. The composition of claim 6, wherein said dodecyl thio-damascone is present in an amount from about 0.01% to about 1.5%, by weight of said composition.

8. The composition of claim 1, wherein said malodor binding polymer is a polyamine having a molecular weight of at least 150 Daltons and 15% to 80% primary amino groups

9. The composition of claim 1, wherein said malodor binding polymer is a homopolymeric polyethyleneimine having a molecular weight of about 1,000 to about 2,000,000 Daltons.

10. The composition of claim 1, wherein said malodor binding polymer is present in an amount of about 0.01% to about 1%, by weight of said composition.

11. The composition of claim 1, wherein said aqueous carrier is present in an amount of 90% to about 99.5%.

12. A freshening composition for reducing malodor comprising:
about 0.02% to about 0.1%, by weight of said composition, of a sulfur-containing pro-perfume;
a perfume material;
at least about 90%, by weight of said composition, of an aqueous carrier;
wherein said composition is essentially free of any material that would soil or stain fabric.

13. The composition of claim 12, wherein said ratio of said sulfur-containing pro-perfume is about 2:1 to about 1:1.

14. The composition of claim 12, wherein said sulfur-containing pro-perfume is a C4-C12 thio-damascone.

15. The composition of claim 12, wherein said sulfur-containing pro-perfume is dodecyl thio-damascone.

16. The composition of claim 15, wherein said dodecyl thio-damascone is present in an amount from about 0.02% to about 0.08%, by weight of said composition.

17. The composition of claim 12, wherein said composition further comprises a buffering agent selected from the group consisting of maleic acid, carboxylic acid, dicarboxcylic acid, N-(2-Acetamido)-2-aminoethanesulfonic acid, and mixtures thereof.

18. The composition of claim 12, wherein said composition comprises a pH from about 5 to about 7.

19. The composition of claim 12, wherein said composition is free of anionic surfactants.

20. The composition of claim 12, wherein said composition comprises no more than 3% surfactant by weight of said composition.

21. A freshening composition for reducing malodor comprising:
a sulfur-containing pro-perfume;
a surfactant;
an effective amount of a malodor binding polymer;
about 90% or greater of aqueous carrier;
wherein the ratio of said sulfur-containing pro-perfume to said surfactant is about 1:1 to about 1:10

22. The composition of claim 21, wherein the ratio is about 1:1 to about 1:6.

23. The composition of claim 21, wherein the ratio is about 1:1 to about 1:4.

24. The composition of claim 21, wherein said sulfur-containing pro-perfume is dodecyl thio-damascone.

25. The composition of claim 24, wherein said dodecyl thio-damascone is present in an amount from about 0.01% to about 1.5%, by weight of said composition.

26. The composition of claim 21 wherein said malodor binding polymer is a polyamine having a molecular weight of at least 150 Daltons and 15% to 80% primary amino groups

27. The composition of claim 21, wherein said malodor binding polymer is a homopolymeric polyethyleneimine having a molecular weight of about 1,000 to about 2,000,000 Daltons.

28. The composition of claim 21, wherein said malodor binding polymer is present in an amount of about 0.01% to about 1%, by weight of said composition.

29. An aqueous freshening composition for reducing malodor comprising:
a homopolymeric polyethyleneimine having a molecular weight of 1,000 to 2,000,000;
a perfume material,
about 0.02% to about 0.08%, by weight of said composition, of a dodecyl thio-damascone;
about 90% to about 99.5%, by weight of said composition, of an aqueous carrier;
wherein said composition is essentially free of any material that would soil or stain fabric.

30. A method of reducing malodor comprising the steps of:
a. providing the freshening composition of claim 1;
b. dispersing an effective amount of said freshening composition onto an inanimate surface or into the air.
